(19) United States
(12) Patent Application Publication
(10) Pub. No.: US 2023/0124876 A1
(43) Pub. Date: Apr. 20, 2023

(54) COMPOSITION OF MICROORGANISMS FOR BREEDING INSECTS

(71) Applicant: MIXSCIENCE, BRUZ (FR)

(72) Inventors: Stéphane THEBAULT, BRUZ CEDEX (FR); Alexandre BRAME, BRUZ CEDEX (FR); Stéphane FROUEL, BRUZ CEDEX (FR)

(73) Assignee: MIXSCIENCE, BRUZ (FR)

(21) Appl. No.: 17/795,764

(22) PCT Filed: Jan. 30, 2021
(86) PCT No.: PCT/EP2021/052224

§ 371 Date: Jul. 27, 2022

(30) Foreign Application Priority Data
Jan. 31, 2020 (FR) 2001006

(51) Int. Cl. A23K 10/18; A23K 50/90; A23K 50/60; A61K 35/742; A61P 15/08

(52) U.S. Cl. CPC A23K 10/18; A23K 50/60; A23K 50/90; A61K 35/742; A61P 15/08

(57) ABSTRACT

A composition for use thereof in the prophylaxis of microbial pathological conditions affecting the growth of insects for breeding, the composition including essentially at least one strain of bacterium of the Bacillus genus, the strain being chosen from: a strain of Bacillus subtilis strain NOL01 as deposited with the CNCM on Mar. 14, 2012 under number CNCM I - 4606, and a strain of Bacillus belonging to the same operational taxonomic unit as the NOL01 strain, or a mixture thereof.

The invention relates to a composition of microorganisms for breeding insects.

Population growth, urbanization and the rise of the middle classes have increased the global demand for food, animal feed and especially protein. By 2030, more than 9 billion people will need to be fed, as will the billions of animals raised each year for food, recreation and as pets.

To meet this growing demand, the traditional production of feed materials, such as soya and fishmeal, must be intensified, raising the question of resource efficiency and the use alternative protein sources.

New protein solutions intended for animal and human food must therefore be sought.

As recently mentioned by major international bodies such as the Food and Agriculture Organization (FAO) and the World Health Organization (WHO), one of the many possible responses to the problems of human and animal food safety is to consider breeding insects, and their consumption and development.

To date, only a small number of insect species have already been domesticated because of their commercial value or that of their by-products, in particular silkworms for their fibers and bees for their honey. Other species are bred to feed pets (mealworms and crickets, for example) or for recreation (bait for fishing).

Insect farming has many advantages.

Insects are ubiquitous and reproduce rapidly. They also have a high growth rate, low feed conversion rate and low environmental impact throughout their life cycle. In addition, insects can feed on fruits or vegetables that have become unfit for human consumption.

Insects are nutritious, with a high content of high-quality proteins, fats and minerals, and are therefore considered a raw material of interest for animal or human nutrition. This nutritional value of insects depends on their life stage (metamorphic stage), their habitat and their diet. However, it is widely accepted that insects provide high-quality proteins and nutrients compared to beef and fish.

More than 1,900 species of insects are currently listed as being edible for humans around the world. However, there is little data on the quantities of insects actually consumed. According to available data, the most commonly consumed insects are beetles (Coleoptera) (31 %), caterpillars (Lepidoptera) (18%), bees, wasps and ants (Hymenoptera) (14%), grasshoppers, locusts and crickets (Orthoptera) (13%), cicadas, leaf beetles and leafhoppers, scale insects and hemipterans (Hemiptera) (10%), termites (Isoptera) (3%), dragonflies (Odonata) (3%), flies (Diptera) (2%) and other categories (5%).

The insects can be eaten whole, or ground into a paste or even in the form of a powder after drying and grinding. These pastes and powders can be added to other foods low in protein to increase their nutritional value (chocolate bars, biscuits, etc.).

However, European regulations on the human consumption of insects or products derived from insects are still uncertain and their application remains uneven under national legislation.

The majority of known edible insect species are harvested from the wild and eaten whole. However, mini-farms, either domestic or local, are developing around the world (e.g. mini-farm supported by a pig farm). These mini-farms offer the possibility of increasing the supply of insect protein without compromising wild insect populations.

However, to meet the challenges mentioned above, more and more companies are working to intensify and industrialize insect farming.

In particular, the black soldier fly ( Hermetica illucens) and the mealworm (Tenebrio molitor) both combine a large number of qualities sought after for insect production on an industrial scale, namely: high growth rate, short development, high egg-laying rate, ability to live in high-density conditions, recovery of green waste, sanitizing power of the substrate.

Like any breeding with a commercial purpose, insect breeding must meet performance, quality, animal welfare and health safety requirements for the user, the consumer and the environment.

In insect farms, it is possible to observe occasional mortalities of individuals or more significant mortalities that can affect an entire batch, sometimes very quickly. Individual mortality can be caused by many things: physiological causes (feeding problem, poor rearing conditions, rearing system poorly adapted to the species), genetic causes (problem of inbreeding, degeneration) or pathological causes.

Regarding pathologies, these can be linked to a microbial infection of the viral, fungal or bacterial type or even to a parasitic infection.

To meet the new expectations of the world food market and the production requirements of the insect sector, in particular taking its specificities into account, it is necessary to improve insect production yields, in particular by securing insect growth and limiting mortality throughout their life cycle.

For example, patent application FR2985664 describes a probiotic composition intended to prevent winter mortality in bees, comprising Bacillus, optionally in combination with other species of bacteria.

Other examples of bacterial compositions are known for example from application CN106854627, which describes a mixture of strains of Kocuria marina FE01, Lysinibacillus boronitolerans FE04, and Proteus mirabilis FE008 for use thereof to optimize the growth of Hermetia illucens during the transformation of chicken manure.

Despite these known compositions of the prior art, the need to provide a composition favorable to the good development of insects for breeding remains.

The invention aims to remedy this deficit.

An object of the invention is to provide a microbial composition, as well as a method for improving the performance of insect farms, in particular black solder fly (BSF) farms.

The objective is to take advantage of the larvae/microbiota relationship in order to improve the conversion efficiency and yield of the production of BSF larvae, i.e. to improve the growth rate of the larvae and the size of the larvae obtained, and to reduce mortality during rearing.

Therefore, the invention relates to a composition for the use thereof to improve the zootechnical performance of insects for breeding, in particular for the prophylaxis or treatment of microbial pathologies affecting said insects for breeding, in particular black soldier flies, said composition consisting essentially of at least one strain of bacteria of the Bacillus genus, said strain being chosen in particular from:
an NOL01 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4606, and
a strain of Bacillus belonging to the same operational taxonomic unit (OTU) as said NOL01 strain,
or a mixture of these strains.

The invention is based on the surprising finding made by the inventors that specific strains of Bacillus, in particular strains of Bacillus belonging to the same OTU as the NOL01 strain, and in particular strains of Bacillus subtilis, are capable, when placed in contact with insects for breeding, of promoting the zootechnical performance of the breeding, in particular to reduce mortality, to increase yield, etc. In addition, this composition makes it possible to promote a microbiota favorable to insects, thus limiting the development of undesirable microorganisms.

In the invention, the term “improve the zootechnical performance” means to improve technical criteria linked to production such as the daily weight gain of individuals (ADG), the weight of individuals at slaughter, the food consumption index or even the viability of insects for breeding and their resistance to sanitary episodes (infection rate, mortality rate), the quality of individuals (their protein and lipid content in particular) and their reproductive capacity. Improving these technical criteria affects the overall yield of the farm and its economic performance.

The composition according to the invention “consists essentially” of said strains of bacteria of the genus Bacillus, which means that the composition contains, as active substance, only strains of bacteria of the genus Bacillus mentioned above. This means that the composition is likely to contain other components, but these additional components do not participate in the technical effect: that is to say, these components alone do not have a significant influence on improving the zootechnical performance of the insect farming, in particular on the treatment or prevention of microbial pathologies affecting the growth of the farmed insects.

In the invention, “strain of bacteria” means all the individuals (bacteria) resulting from successive subcultures of a bacterial colony, this set being essentially homogeneous, that is to say, the very great majority of bacteria that make up the strain have the same genotype.

In other words, a strain is a part of a bacterial species that differs from other bacteria of the same species by a minor but identifiable difference. A strain is also defined as a population of bacteria that descended from a single organism or pure isolate culture. Strains of the same species may differ slightly from each other in many ways.

Advantageously, the invention relates to the aforementioned composition for the use thereof as defined above, wherein said at least one strain of bacterium of the genus Bacillus, in particular Bacillus subtilis, is chosen from:
an NOL01 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4606, and
a strain of Bacillus, in particular Bacillus subtilis, whose sequence encoding the 16S rRNA has at least 97% identity with the sequence encoding the 16S rRNA of the NOL01 strain,
or a mixture of these strains. The inventors have shown that an NOL01 strain or the strains having a genome very similar to this one are capable, by a simple treatment, of preventing and/or treating the microbial pathologies of insects for breeding and of improving their zootechnical performance. More particularly, the inventors have been able to demonstrate that the NOL01 strain, and the strains of Bacillus, in particular Bacillus subtilis, having a very similar genome are capable of preventing or treating microbial pathologies affecting livestock insects or even of improving the zootechnical performance of said insects for breeding.

In the invention, when reference is made to a strain deposit with the CNCM, reference is made to a deposit made with the Collection Nationale de Cultures de Microorganismes [National Collection of Microorganism Cultures] (CNCM, Institut Pasteur 25-28 rue du Docteur Roux 75724 Paris, France) according to the Budapest Treaty on the International Recognition of the Deposit of Microorganisms for the Purposes of Patent Procedure.

As mentioned above, the composition according to the invention essentially consists of an NOL01 strain or a similar strain, or a mixture thereof. This means that the composition according to the invention consists essentially of said NOL01 strain, or consists essentially of one or more strains belonging to the same operational taxonomic unit (OTU) as the NOL01 strain, or else said NOL01 strain and one or more strains belonging to the same operational taxonomic unit (OTU) as the NOL01 strain. Examples will be given below.

In the invention, “a Bacillus strain belonging to the same operational unit (OTU) as said NOL01 strain” or “a similar strain” to NOL01 , or “strains having a very similar” or “very close” genome to NOL01, means all strains of Bacillus, in particular Bacillus subtilis, having at least 97% of genomic sequences similar to the sequence of genes encoding the 16S rRNA of the NOL01 strain.

In biology, the operational taxonomic unit (OTU) is the basic unit of phylogenetic analysis used to group phylogenetically close individuals. The DNA sequences of a given gene, which then serves as a taxonomic marker, are grouped together by data partitioning (clustering) according to their identity. One gene that is primarily used (reference gene) is 16S rRNA for bacteria. Thus, the OTUs are defined on the basis of an identity threshold chosen by those skilled in the art, which is commonly 97%.

In the invention, the term “insects for breeding” means bees including the Asian bee ( Apis cerana), the European honey bee ( Apis melifera), the dwarf honey bee ( Apis florea), spiders including the black tarantula spider (Brachypelma smithi), caterpillars including the caterpillar Bunaeopsis spp, ladybugs including the 7-spot ladybug ( Coccinella septempunctada), locusts including Locusta migratoria and the desert locust ( Schistocerca gregaria), ants including the ant of Colombia ( Attalaevigata), crickets including the field cricket ( Gryllus assimilis), the house cricket ( Acheta domestica), the tropical house cricket ( Gryllodes sigillatus), flies including the house fly ( Musca domestica), the black soldier fly ( Hermetia illucens), mealworms including the lesser mealworm ( Alphitobius diaperinus), the mealworm ( Tenebrio molitor), bedbugs including the giant water bug ( Lethocerusamericanus), beetles including the rhinoceros beetle ( Allomyrina dichotoma), scorpions including the black scorpion ( Heterometrus longimanus), termites including Mastodermes darwiniensis, worms including the silkworm ( Bombyx mori), the bamboo worm ( Omphisa fuscidentalis), the palm weevil ( Rhynchophorus ferrugineus), the superworm ( Zophobas morio).

The advantageous insects for breeding according to the invention are black soldier flies (BSF) or else Hermetia illucens.

In the invention, the term “microbial pathologies affecting said insects for breeding” means all the pathologies linked to the infection of breeding by entomopathogens and affecting the growth, development, molting and/or survival of the insects for breeding.

The death of the host insect occurs by the action of toxins emitted by certain germs ( Bacillus thuringiensis), by bacterial septicemia ( Bacillus popilliae), following the lysis of cells of the intestinal epithelium or by destruction of fat or blood cells (viruses, protozoa).

Most entomopathogenic germs are specific. However, non-specific bacteria can become pathogenic when rearing conditions become abnormal; thus, the enterobacterium Serratia marcescens Bizio, Pseudomonas or even cocci and streptococci bacteria can be harmful and responsible for mortality in farmed insects. Furthermore, protozoa, such as coccidia or microsporidia, can kill their host at any stage of the development cycle, or considerably reduce the life of the adults, their fecundity as well as the fertility of the eggs.

It is particularly advantageous in the invention for the aforementioned composition to comprise at least two strains of bacteria of the genus Bacillus, and in particular at least two different strains of Bacillus.

It is even more advantageous in the invention for the aforementioned composition to comprise at least three strains of bacteria of the genus Bacillus, and in particular at least three different strains of Bacillus.

Advantageously, the invention relates to the composition for the use thereof as defined above, wherein said strain of bacterium of the genus Bacillus, in particular Bacillus subtilis, belonging to the same operational taxonomic unit (OTU) as said NOL01 strain is chosen from:
an NOL02 strain as deposited with the CNCM on Jan. 21, 2016 under number CNCM I - 5043, and
an NOL03 strain as deposited with the CNCM on Mar. 14, 2012 under number CNCM I - 4607.

The NOL02 and NOL03 strains have a very great phylogenetic proximity to the NOL01 strain, insofar as they are all three parts of the same phylogenetic branch, when their DNA, in particular the DNA encoding the 16S rRNA, is compared.

Advantageously, the invention relates to the composition for the use thereof as defined above, said composition consisting essentially of:
the NOL01 strain, or
the NOL02 strain, or
the NOL03 strain, or
the NOL01 strain and the NOL02 strain, or
the NOL01 strain and the NOL03 strain, or
the NOL02 strain and the NOL03 strain, or
the NOL01 strain, the NOL02 strain and the NOL03 strain.

Advantageously, the invention relates to the composition for the use thereof as defined above, wherein said composition comprises from 10⁴ to 10¹¹ live bacterial colonies of said Bacillus or Bacilli, in particular Bacillus subtilis, the bacterial colonies being per mL or g of composition.

In other words, in this advantageous embodiment, if the composition according to the invention is in liquid form, said composition will comprise from 10⁴ to 10¹¹ bacterial colonies of Bacillus, in particular Bacillus subtilis, per mL of composition, for each of the strains when the composition comprises at least two strains.

If, on the other hand, the composition according to the invention is in dehydrated or non-aqueous form (in solid form), said composition will comprise from 10⁴ to 10¹¹ bacterial colonies of Bacillus per g of composition, for each of the strains when the composition comprises at least two strains.

In the invention, “from 10⁴ to 10¹¹ bacterial colonies” means: approximately 10⁴, approximately 5.10⁴, approximately 10⁵, approximately 5.10⁵, approximately 10⁶, approximately 5.10⁶, approximately 10⁷, approximately 5.10⁷, approximately 10⁸, approximately 5.10⁸, approximately 10⁹, approximately 5.10⁹, approximately 10¹⁰, approximately 5.10¹⁰ or approximately 10¹¹ bacterial colonies.

Bacterial colonies are per mL (or gram) of bacterial culture. A person skilled in the art easily knows how to determine this number of bacteria, in particular by counting either manually (using a Malassez blade), or by using an automatic cell counter, or by dilution, then inoculation on agar and counting, in particular visual, of the colonies, or by measuring the optical density.

Advantageously, the invention relates to the composition for the use thereof as defined above, wherein said one or more strains of bacteria of the genus Bacillus, in particular Bacillus subtilis, are in vegetative or spore form, or both.

In the advantageous embodiments of the invention wherein the aforementioned composition essentially consists of two strains of bacteria of the genus Bacillus, in particular Bacillus subtilis, belonging to the same operational taxonomic unit (OTU) as said NOL01 strain, said Bacilli are either all in vegetative form, or all in spore form, or one strain is in spore form and the other strain is in vegetative form.

In the advantageous embodiments of the invention wherein the aforementioned composition essentially consists of three strains of Bacillus, in particular Bacillus subtilis, belonging to the same operational taxonomic unit (OTU) as said NOL01 strain, either these three strains are in spore form, or these three strains are in vegetative form, or one strain is in vegetative form and the other two are in spore form, or one strain is in spore form and the other two are in vegetative form. If the composition comprises more than three strains of bacteria as defined above, those skilled in the art will understand which combinations are applicable.

In addition, the same strain of bacteria according to the invention can be in the vegetative form, or in the spore form, or form a mixture of bacteria in the vegetative form and in the spore form.

Those skilled in the art will know which form is the most suitable for each of the strains used in the invention.

A composition is also described for the use thereof as defined above, in combination with at least one strain of lactic acid bacteria, in particular at least one strain of Lactococcus lactis.

The inventors have observed that the composition according to the invention can be supplemented with at least one strain of lactic acid bacteria, in particular at least one strain of Lactococcus lactis, as an alternative composition to the prior art.

The completed composition described above comprises at least two strains of bacteria of the genus Bacillus, and in particular at least two different strains of Bacillus, or even at least three strains of Bacillus, and at least one strain of lactic acid bacteria, in particular of Lactococcus lactis.

The strain of lactic acid bacteria is in particular a strain of lactic acid bacteria NOL11 as deposited with the CNCM on Mar. 14, 2012 under number CNCM I -4609.

Therefore, described compositions are the following compositions for their use as defined above:
the NOL01 strain, in combination with the NOL11 strain, or
the NOL02 strain, in combination with the NOL11 strain, or
the NOL03 strain, in combination with the NOL11 strain, or
the NOL01 strain and the NOL02 strain, in combination with the NOL11 strain, or
the NOL01 strain and the NOL03 strain, in combination with the NOL11 strain, or
the NOL02 strain and the NOL03 strain, in combination with the NOL11 strain, or
the NOL01 strain, the NOL02 strain and the NOL03 strain, in combination with the NOL11 strain.

Said at least one strain of lactic acid bacteria is possibly present at a rate of 10⁴ to 10¹¹ live bacterial colonies, the bacterial colonies being per mL or g of composition.

The definition of the quantity of bacteria of the genus Bacillus, in particular Bacillus subtilis, mentioned above applies mutatis mutandis to the strain of lactic acid bacteria.

The aforementioned composition for the use thereof as previously defined can be applied, or can be in a form to be, or capable of being, applied, to the eggs, the larvae, the prepupa, the pupae or the adult individuals of the insects for breeding, directly or indirectly via the rearing substrate, including drinking water. As regards water for watering, this can be sprayed or misted on the eggs, larvae, prepupa, pupae or adult individuals of insects for breeding or on the walls of the breeding enclosures of said eggs, larvae, prepupa, pupae or adult individuals of the insects for breeding.

The composition can also be applied to the supports, containers or any materials in contact with the eggs, the larvae, the prepupae, the pupae or the adult individuals of the insects for breeding.

Advantageously, the invention relates to the aforementioned composition for the use thereof as defined above, said composition being applied to the eggs, larvae, prepupae, pupae or adult individuals of the insects for breeding. The objective is for the insects, at one or more stages of their development, to be in direct contact with the composition according to the invention so that the pathogens cannot affect their growth, and for as many as possible of them to be able to reach the pupa or even adult stage depending on the production.

Advantageously, preference will be given to early direct contact of the insects with the composition according to the invention.

Thus, is it possible to apply the composition according to the invention directly to the eggs.

The invention also relates to insect food comprising a composition as defined above for the prophylaxis or treatment of microbial pathologies affecting said insects for breeding, that is to say, microbial pathologies affecting growth, development, molting and/or survival of insects for breeding. The invention also relates to the use of said food for improving the zootechnical performance of insects for breeding.

It is particularly advantageous for the composition according to the invention to be integrated into food allowing the growth of insects at their various stages of development, in particular for the nutrition of the larvae after hatching and before pupation.

The food according to the invention therefore consists of nutritional supports conventionally used for insects for breeding, for example food by-products, such as peelings of fruits or vegetables such as bananas, carrots, broccoli, zucchini, cereals (wheat, corn, etc.), bran and spent grains.

The aforementioned food allows:
bringing the composition according to the invention into indirect contact with insects at their various stages of development, insofar as the insects develop in their growth substrate,
absorption of the bacteria constituting the composition according to the invention, during the consumption of the nutritional substrate, or else
modification of the food by the bacteria of the composition, so that the nutrients resulting from the action of said bacteria are more easily bioavailable for the insects for breeding.

Even more advantageously, the composition according to the invention is present in the aforementioned food at the rate of 100 g to 1 kg per ton of food.

In another aspect, the invention relates to the use of a composition consisting essentially of at least one strain of bacteria of the Bacillus genus, said strain being chosen in particular from:
an NOL01 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4606, and
a strain of Bacillus belonging to the same operational taxonomic unit (OTU) as said NOL01 strain,
or a mixture of these strains,
for improving the zootechnical performance of insects for breeding, in particular black soldier flies.

Advantageously, the invention relates to the aforementioned use, wherein said Bacillus strain belonging to the same OTU as said NOL01 strain is chosen from:
a strain of Bacillus subtilis strain NOL02 as deposited with the CNCM on Jan. 21, 2016 under number CNCM I - 5043, and
a strain of Bacillus subtilis strain NOL03 as deposited with the CNCM on Mar. 14, 2012 under number CNCM I - 4607.

Even more advantageously, the invention relates to the aforementioned use, said composition consisting essentially of:
the NOL01 strain, or
the NOL02 strain, or
the NOL03 strain, or
the NOL01 strain and the NOL02 strain, or
the NOL01 strain and the NOL03 strain, or
the NOL02 strain and the NOL03 strain, or
the NOL01 strain, the NOL02 strain and the NOL03 strain.

According to the aforementioned use, it is advantageous for the composition to comprise from 10⁴ to 10¹¹ live bacterial colonies of said Bacillus or Bacilli, the bacterial colonies being per mL or g of composition.

Advantageously, the invention relates to the use as defined above for improving zootechnical performance, said composition being applied to the eggs, larvae, prepupae, pupae or adult individuals of the insects for breeding.

According to the aforementioned use, it is also even more advantageous for said one or more strains of bacteria of the genus Bacillus to be in vegetative form or in spore form, or both.

Mention is also made of the aforementioned use, said composition being used in combination with at least one strain of lactic acid bacteria, in particular a strain of Lactococcus lactis, in particular a strain of lactic acid bacteria NOL11 as deposited with the CNCM on Mar. 14, 2012 under number CNCM I - 4609, in particular at a rate of 10⁴ to 10¹¹ live bacterial colonies, the bacterial colonies being per mL or g of composition.

Also described is the use as defined above, said composition being applied or in a form capable of being applied, directly or indirectly via the rearing substrate, to the eggs, larvae, prepupa, pupae or adult individuals of insects for breeding.

Provision is also made for the use of the aforementioned food, to improve the zootechnical performance of insects for breeding, in particular for the prophylaxis or treatment of microbial pathologies affecting said insects for breeding.

A composition for the use thereof to improve the zootechnical performance of insects for breeding is further disclosed here, in particular for the prophylaxis or treatment of microbial pathologies affecting said insects for breeding, in particular black soldier flies, said composition consisting essentially of at least one strain of lactic bacteria, in particular Lactococcus lactis, said strain being chosen in particular from:
an NOL11 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4609, and
a strain of lactic acid bacteria belonging to the same operational taxonomic unit (OTU) as said NOL11 strain,
or a mixture of these strains.

The composition “consists essentially” of said strains of lactic bacteria, which means that the composition contains, as active substance, only strains of lactic bacteria. This means that the composition is likely to contain other components, but these additional components do not participate in the technical effect: that is to say, these components alone do not have a significant influence on improving the zootechnical performance of the insect farming, in particular on the treatment or prevention of microbial pathologies affecting the growth of the farmed insects.

It may be particularly advantageous for the genetic proximity of the lactic acid bacteria to be more than 97% genome identity, relative to the 16S rRNA of the NOL11 strain.

The abovementioned composition for its abovementioned use is such that it can comprise from 10⁴ to 10¹¹ live bacterial colonies of said strain(s) of lactic acid bacteria, the bacterial colonies being per mL or g of composition.

The composition may also be in combination with at least one strain of bacteria of the Bacillus genus, in particular at least one strain of Bacillus subtilis.

The disclosure also relates to an aforementioned composition for its aforementioned use, wherein said strain of Bacillus is chosen in particular from:
an NOL01 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4606, and
a strain of Bacillus belonging to the same operational taxonomic unit (OTU) as said NOL01 strain,
or a mixture of these strains.

The Bacillus strain belonging to the same OTU as said NOL01 strain is chosen in particular from:
a strain of Bacillus subtilis strain NOL02 as deposited with the CNCM on Jan. 21, 2016 under number CNCM I - 5043, and
a strain of Bacillus subtilis strain NOL03 as deposited with the CNCM on Mar. 14, 2012 under number CNCM I - 4607.

This means that the aforementioned composition for its aforementioned use, consisting essentially of at least one strain of Bacillus, is chosen from:
the NOL01 strain, or
the NOL02 strain, or
the NOL03 strain, or
the NOL01 strain and the NOL02 strain, or
the NOL01 strain and the NOL03 strain, or
the NOL02 strain and the NOL03 strain, or
the NOL01 strain, the NOL02 strain and the NOL03 strain.

The composition comprises in particular from 10⁴ to 10¹¹ live bacterial colonies of said Bacillus or Bacilli, the bacterial colonies being per mL or g of composition.

The composition is further in particular applied, or can be in a form to be, or capable of being, applied, to the eggs, the larvae, the prepupa, the pupae or the adult individuals of the insects for breeding, directly or indirectly via the rearing substrate.

The use of a composition consisting essentially of at least one strain of lactic acid bacteria is also described, said strain being chosen in particular from:
an NOL11 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4609, and
a strain of lactic acid bacteria belonging to the same operational taxonomic unit (OTU) as said NOL11 strain,
or a mixture of these strains,
to improve the zootechnical performance of insects for breeding, in particular for the prophylaxis or treatment of microbial pathologies affecting said insects for breeding, in particular black soldier flies.

The composition may comprise from 10⁴ to 10¹¹ bacterial colonies of said lactic acid bacteria, the bacterial colonies being per mL or g of composition.

In the context of the use described above, said composition is in combination with at least one strain of bacteria of the genus Bacillus, in particular at least one strain of Bacillus subtilis, in particular
an NOL01 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4606, and
a strain of Bacillus belonging to the same operational taxonomic unit (OTU) as said NOL01 strain,
or a mixture of these strains.

The invention will be better understood in the light of the following figures and drawings.

### BRIEF DESCRIPTION OF THE FIGURES

FIG. 1 is a box plot representing the individual weights of a representative sample of 30 larvae per fattening tank (in g) for the control batches (Control, D1+H₂O and D3+H₂O - B) compared with those of the test batches (D1+CI and D3+CI - A), on D14. t-test: p-value = 0.0002434.

FIG. 2 is a box plot representing the masses of pupae per fattening tank (in kg) for the control groups (Control, D1+H₂O and D3+H₂O - B) compared to those of the test groups (D1+CI and D3+CI - A), on sorting day D28. t-test: p-value = 0.001243.

FIG. 3 is a box plot representing the survival rates per fattening tank (in %) for the control groups (Control, D1 +H₂O and D3+H₂O - B) compared to those of the test groups (D1+CI and D3+CI - A), on sorting day D28. t-test: p-value = 0.03293.

FIG. 4 is a box plot representing the estimated number of pupae per fattening tank for the control groups (Control, D1+H₂O and D3+H₂O - B) compared to that of the test groups (D1+CI and D3+CI - A), on sorting day D28.

### EXAMPLES

### Example 1

The objective of this test is to demonstrate the effect of a composition according to the invention (Cl) on the increase in production yields of BSF larvae for breeding.

This test shows the results obtained under experimental conditions on 30 batches divided into 5 modalities (i.e. 6 repetitions per modality).

### 1/ Material and Methods

The 5 modalities vary according to 2 criteria:
Spraying of a composition according to the invention or not: the control groups undergo either no spraying (Control) or a spraying of water (Dx+H₂O); the test groups are sprayed with a composition according to the invention (Dx+CI),
Spraying frequency: when spraying occurs, it is carried out once a day (D1+CI and D1+ H₂O) or once every 3 days (D3+CI and D3+ H₂O).

The substrate, common to the 5 modalities, is composed of plant raw materials that are ground (using a grinder) in order to mix together the raw materials (fruit or vegetable peelings such as bananas, carrots, broccoli, zucchini, cereals (wheat, corn, etc.)) in such a way that the substrate is nutritionally suitable to meet the average nutritional needs of an insect larva.

Upstream, the eggs come from nest boxes and are collected daily. On D0, they are placed in incubation tanks with a capacity of 300 g of substrate (average pH 5.8) for incubation (30° C. - 70% humidity) for 6 days. Approximately 0.100 g of BSF eggs are thus cultured, corresponding approximately to 8 clutches, in each of the 30 incubation tanks. The average number of inoculated eggs per incubation tank is 5200 eggs.

On the 7th day (D7), the young larvae and their substrate are transferred to 30 fattening tanks to continue their development, to which 8 kg of fresh substrate (mean pH 5.9) is added.

The fattening tanks are then placed in the production area.

On D28, the test ends with individuals at the pupal stage.

The composition according to the invention sprayed for the D1+CI and D3+CI groups consists of the following three strains of Bacillus subtilis: NOL01 , NOL02 and NOL03.

The composition according to the invention is supplied frozen for breeding and packaged in a 20 mL bottle. After thawing, the 20 mL vial is diluted in 1 liter of water in the reservoir of a sprayer and the preparation is stored at 4° C. until use. The reservoir is shaken before each daily use or every 3 days.

For the groups with spraying, spraying is carried out per incubation or fattening tank (i.e. approximately 2 mL), either of the composition according to the invention, or of water without composition according to the invention from D1 to D17 (which corresponds to 17 sprays of approximately 2 mL per container for batches D1+CI and D1+H20 and 7 sprays of approximately 2 mL per container for batches D3+CI and D3+H20).

The data is collected:
every day: recording of the temperature of the incubation or fattening tanks (in °C)
at D14 and D17, mass of larvae (in g)
at sorting = D28, mass of pupae (in g).

### 2/ Results and Conclusions

The results are presented in FIGS. 1 to 4.

FIG. 1 shows an individual average weight of the larvae of the test batches (D1+CI and D3+CI) significantly higher than that of the control groups (Control, D1+H₂O and D3+H₂O) on D14 after spraying.

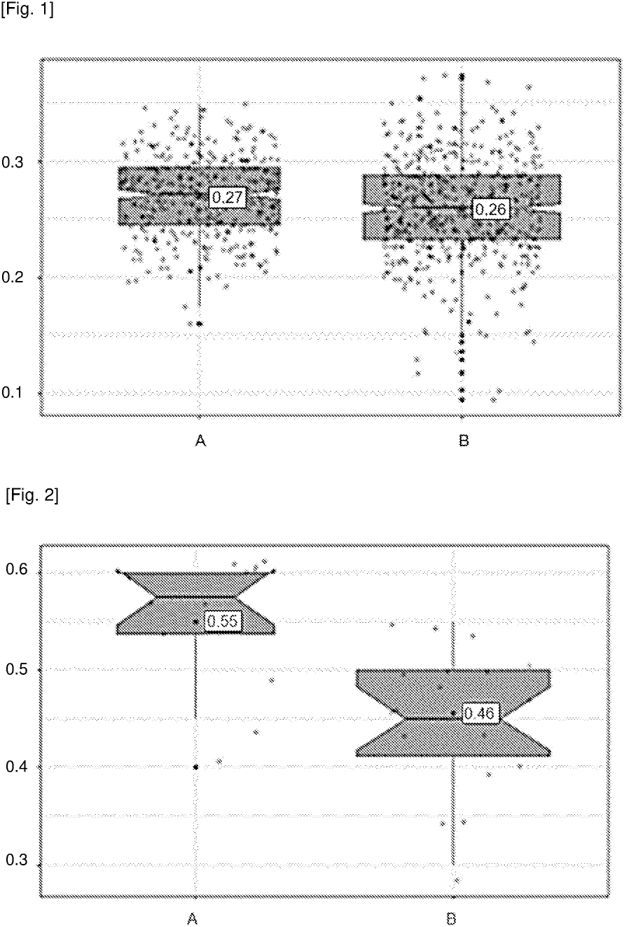

FIG. 2 shows an average mass of the pupae of the test groups (D1+Cl and D3+CI) significantly higher than that of the control groups (Control, D1+H₂O and D3+H₂O) on D28.

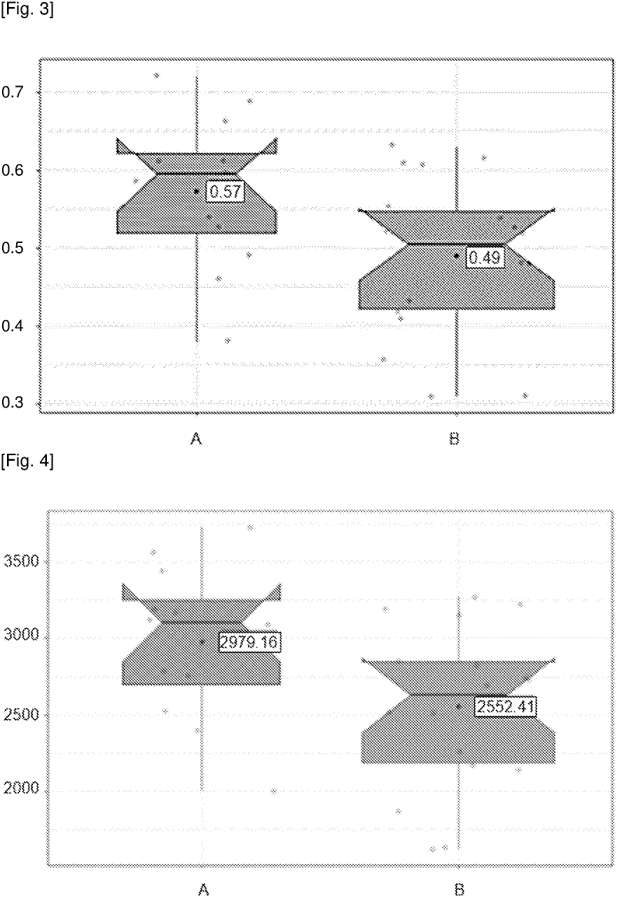

FIG. 3 shows an average survival rate for the test groups (D1+Cl and D3+CI) significantly higher (+0.08 points) than that of the control groups (Control, D1+H₂O and D3+H₂O) at D28.

FIG. 4 shows a greater average number of pupae at D28 in the test tanks than the control tanks, which reflects a lower mortality rate for the test groups (reduction in mortality).

These data show that the supply of the composition according to the invention via the substrate has a beneficial effect on the weight gain and the final number of individuals (reduced mortality) of the groups of BSF treated, whether daily or every 3 days. This results in a significant gain in final biomass.

### Example 2

The objective of this test is to demonstrate the effect of a composition according to the invention on the production of BSF larvae for breeding in the event of a bacterial pathology harmful to production.

This test shows the results obtained under experimental conditions on 40 batches divided into 5 modalities (i.e. 8 repetitions per modality) during an unexpected episode of bacterial contamination of the farm.

### 1/ Material and Methods

As in example 1, these 5 modalities vary according to 2 criteria, namely the spraying on the rearing substrate of a composition according to the invention or not (absence of spraying, spraying of a composition according to the invention or spraying of H₂O) and the frequency of this spraying (daily or every 3 days).

The incubation and fattening of the 40 batches is identical to example 1.

The composition of the substrate common to the 5 modalities is identical to that of example 1.

The composition according to the invention sprayed for groups D1+CI and D3+CI and its implementation are identical to those of example 1 apart from the number of sprays per tank.

Thus, for the groups with spraying, 10 sprayings are carried out per incubation or fattening tank (i.e. approximately 10 mL), either of the composition according to the invention, or of water without composition according to the invention, on a daily basis or every 3 days depending on the batch.

### 2/ Incident

On D11, significant mortality was observed in certain batches of the test due to contamination of the farm by a pathogenic bacterium, Bacillus thuringiensis, commonly used in organic farming as a natural insecticide. This contamination affected all of the 40 tanks in the test, both the test batch tanks (D1+CI and D3+CI) and the control batch tanks (Control, D1+H₂O and D3+H₂O).

### 3/ Results

From D0 to D14, no difference in temperature or pH change was observed in the fattening tanks of the test batches (D1+CI and D3+CI) versus the control batches (Control, D1+H₂O and D3+H₂O).
On D14, no significant difference was observed between the mass of the larvae of the different lots according to the “Frequency of spraying” modality, daily or every 3 days.
On D14, a significantly positive difference (+15.4%) was observed in the average mass of the larvae of the test lots (D1 +Cl and D3+CI) compared to that of the control lots (Control, D1+H₂O and D3+ H₂O).
It was observed that the mortality did not occur at the same speed in all the tanks, the fattening tanks with spraying of the composition according to the invention having taken approximately 3 days longer to exhibit the pathology than the tanks without spraying (Control) or with water spraying (D1+H₂O and D3+H₂O). The appearance of the first symptoms of the pathology in the Test tanks led to the test being stopped on D14.
The larvae of the Control lots, that is to say, without spraying the composition according to the invention or with spraying water, showed a higher and earlier mortality rate (from D11) than the test lots.

### 4/ Conclusions

Despite bacterial contamination causing high mortality of the larvae, the application of the composition according to the invention makes it possible to continue fattening of the BSF larvae for 3 days longer compared to the control lots: these 3 additional days have a real economic interest for an insect protein production sector due to the continued growth of the treated larvae. Indeed, except in the context of rearing for reproduction, the larvae are slaughtered for recovery in oil or meal before the prepupal phase, around D14.

### Example 3

The objective of this test is to compare the effect of different compositions on the production yields of BSF larvae for breeding, and therefore on zootechnical performance.

This test shows the results obtained under experimental conditions on 72 lots divided into 3 modalities and 2 batches.

### 1/ Material and Methods

The 3 modalities vary according to the composition of the substrate used:
Control T-: common substrate (without addition);
L: common substrate + composition consisting of the strain of lactic acid bacteria Lactococcus lactis spp lactis 1 strain NOL11, said strain being deposited with the CNCM under number CNCM I - 4609;
B+L: common substrate + composition consisting of the combination of strains NOL01, NOL02, NOL03 and NOL11.

Table 1 presents the test device, in number of lots per modality, according to the production batch considered:

TABLE 1

| Modality | T- | B. lactic (L) | B+L |
|---|---|---|---|
| Batch 1 | 18 | 18 | |
| Batch 2 | 18 | | 18 |
| Total | 36 | 18 | 18 |

The composition of the substrate common to the 3 modalities is identical to that of example 1.

Bacterial compositions L and L+B are added to this common substrate, for lots L and L+B, up to 50 g per 100 kg of substrate. The bacterial compositions are provided in powder form and incorporated into the substrate at the time of its preparation according to conventional techniques known in the art.

The incubation of the 72 lots differs from that of example 1 on the following points:
250 g of substrate per incubation tank
15 laying units per incubation tank

Measurements to take at start-up and during incubation:
The pH, density and dry matter are recorded during the manufacture of each of the different substrates (D0).
The temperature and relative humidity of the incubator are recorded daily.
The weight of the incubation trays is also recorded daily.
On leaving incubation (D7), the trays are weighed one last time, the mass of 30 larvae is recorded before transfer to the fattening tanks.

On D7, the incubation tanks are transferred to fattening tanks containing 15 kg of fresh substrate. The fattening tanks are then placed in the production area and kept in optimal breeding conditions until harvest day on D17.

Measurements to take during fattening:

The temperature and the relative humidity of the fattening area are recorded daily as well as the temperature inside the tanks, the mass of 30 larvae and an observation of good health. The pH of each tank is measured on D10, D13, D15 and on harvest day (D17).

Measurements to be taken on harvest day at D17:

On harvest day (D17), each tank is subject to the following measurements:
The mass of the tank before sorting (in Kg)
The mass of larvae collected (in Kg)
The mass of amendment harvested (in Kg)
The mass of 30 larvae.

On harvest day D17, the results concerning the individual weight, the survival rate of the larvae and the mortality are recorded and are expressed as a percentage relative to the control lot T-.

Table 2 shows all the results of this example:

TABLE 2

| Strains | Individual weight | Survival | Yield of larvae |
|---|---|---|---|
| Lactococcus | + 25%*** | - 34%*** | - 18%*** |
| Lactococcus and Bacillus | - 3% | + 11% | + 9.2% |

The data are expressed as a percentage relative to the control T- (common substrate).
***: significant variation compared to the control (p value <0.05)

The results obtained during this test evaluating the improvement of zootechnical performance show quite surprising results:
i) it is noted that the individual weight is increased in the Lactococcus lots, but that this significant difference is no longer observed when the two bacterial types are used simultaneously,
ii) survival significantly collapses by more than a third in the Lactococcus lots, and it does not vary significantly from the control in the Bacillus + Lactococcus lots, and
iii) the yield of the larvae is significantly reduced in the Lactococcus lots and is unchanged in the Bacillus + Lactococcus lots compared with the control.

Table 3 shows all the results of the three previous examples:

TABLE 3

| Strains | Tests | Individual weight | Survival | Yield of larvae |
|---|---|---|---|---|
| Bacillus | Example 1: Example 2: | + 3.45% + 13.4%*** | + 17%*** 3 more days*** | + 20.7%*** n/d |
| Lactococcus | Example 3: | + 25%*** | - 34%*** | - 18%*** |
| Lactococcus and Bacillus | Example 3: | - 3% | + 11% | + 9.2% |

The data are expressed as a percentage relative to the control T- (common substrate)
n/d: not determined
***: significant variation compared to the control (p value <0.05)

These three examples show that, very surprisingly, the sole use of a substrate comprising Bacillus strains according to the invention allows:
increased/improved zootechnical performances, evaluated on the basis of the survival of the breeding, its yield and the weight of the individuals, whereas
the use of strains of Lactococcus has a predominantly detrimental effect on said performance.

Indeed, although the individual weight of the larvae is significantly better in this last lot, this is linked to the fact that a high mortality occurred and therefore that the remaining larvae had more substrate to gain weight (less density per substrate unit).

Furthermore, the combination of the two species of bacteria ( Bacillus subtilis and Lactococcus lactis) does not make it possible to improve the performance obtained with the Bacillus strains alone, these latter results being comparable to those of the control (no bacteria).

Therefore, a composition according to the invention indeed shows particularly interesting and surprising properties on zootechnical performance as demonstrated by these tests.

1-15. (canceled)

16. A method for improving the zootechnical performance of insects for breeding, comprising a step of administering a composition to the insects,
said composition consisting essentially
of a strain of bacteria of the Bacillus genus, said strain being chosen from:
an NOL01 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4606, and
a strain of Bacillus belonging to the same operational taxonomic unit (OTU) as said NOL01 strain,
or a mixture of these strains.

17. The method according to claim 16, wherein the insects for breeding are black soldier flies.

18. The method according to claim 16, wherein strain of Bacillus belonging to the same operational taxonomic unit (OTU) as said NOL01 strain is chosen from:
a strain of Bacillus subtilis strain NOL02 as deposited with the CNCM on Jan. 21, 2016 under number CNCM I - 5043, and
a strain of Bacillus subtilis strain NOL03 as deposited with the CNCM on Mar. 14, 2012 under number CNCM I - 4607.

19. The method according to claim 16, said composition consisting essentially of one of:
the NOL01 strain,
the NOL02 strain,
the NOL03 strain,
the NOL01 strain and the NOL02 strain,
the NOL01 strain and the NOL03 strain,
the NOL02 strain and the NOL03 strain, and
the NOL01 strain, the NOL02 strain and the NOL03 strain.

20. The method according to claim 16, wherein said composition comprises from 10⁴ to 10¹¹ live bacterial colonies of said Bacillus or Bacilli, the bacterial colonies being per mL or g of composition.

21. The method according to claim 16, wherein said bacteria of the genus Bacillus are in vegetative form or in spore form, or both.

22. The method according to claim 16, said composition being in a form capable of being applied to the eggs, the larvae, the prepupa, the pupae or the adult individuals of the insects for breeding, directly or indirectly via the substrate.

23. An insect food comprising a composition said composition consisting essentially
of a strain of bacteria of the Bacillus genus, said strain being chosen from:
an NOL01 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4606, and
a strain of Bacillus belonging to the same operational taxonomic unit (OTU) as said NOL01 strain,
or a mixture of these strains.

24. A method improve the zootechnical performance of insects for breeding, the method comprising contacting insects for breeding with the insect food as defined in claim 23.

25. A method for the prophylaxis or treatment of microbial pathologies affecting insects for breeding, in particular black soldier flies, comprising a step of contacting the insects for breeding with a composition,
said composition consisting essentially
of a strain of bacteria of the Bacillus genus, said strain being chosen from:
an NOL01 strain as deposited with the Collection Nationale de Cultures de Microorganismes [National Collection of Microorganism Cultures] (CNCM, Institut Pasteur 25-28 rue du Docteur Roux 75724 Paris, France) according to the Budapest Treaty on Mar. 14, 2012 under number CNCM I - 4606, and
a strain of Bacillus belonging to the same operational taxonomic unit (OTU) as said NOL01 strain,
or a mixture of these strains.

26. The method according to claim 25, wherein said Bacillus strain belonging to the same OTU as said NOL01 strain is chosen from:
a strain of Bacillus subtilis strain NOL02 as deposited with the CNCM on Jan. 21, 2016 under number CNCM I - 5043, and
a strain of Bacillus subtilis strain NOL03 as deposited with the CNCM on Mar. 14, 2012 under number CNCM I - 4607.

27. The method according to claim 24, said composition consisting essentially of one of:
the NOL01 strain,
the NOL02 strain,
the NOL03 strain,
the NOL01 strain and the NOL02 strain,
the NOL01 strain and the NOL03 strain,
the NOL02 strain and the NOL03 strain, and
the NOL01 strain, the NOL02 strain and the NOL03 strain.

28. The method according to claim 25, wherein said composition comprises from 10⁴ to 10¹¹ live bacterial colonies of said Bacillus or Bacilli, the bacterial colonies being per mL or g of composition.

29. The method according to claim 25, wherein said one or more strains of bacteria of the genus Bacillus are in vegetative form or in spore form, or both.

30. The method according to claim 25, said composition being in a form capable of being applied to the eggs, the larvae, the prepupa, the pupae or the adult individuals of the insects for breeding, directly or indirectly via the substrate.

31. A for the prophylaxis or treatment of microbial pathologies affecting said insects for breeding, the method comprising contacting insects for breeding with the insect food as defined in claim 23.
